(19) United States
(12) Patent Application Publication
(10) Pub. No.: US 2008/0255410 A1
(43) Pub. Date: Oct. 16, 2008

(54) CAPSULE ENDOSCOPE AND MANUFACTURING METHOD THEREOF

(76) Inventors: Koji Okuzumi, Akishima-shi (JP); Jun Matsumoto, Hino-shi (JP); Takeaki Nakamura, Hino-shi (JP)

Correspondence Address: OSTROLENK FABER GERB & SOFFEN, 1180 AVENUE OF THE AMERICAS, NEW YORK, NY, 100368403, US

(21) Appl. No.: 12/141,201

(22) Filed: Jun. 18, 2008

Related U.S. Application Data
(63) Continuation of application No. PCT/JP2006/325245, filed on Dec. 19, 2006.

(30) Foreign Application Priority Data
Dec. 19, 2005 (JP) 2005-365209

(51) Int. Cl. A61B 1/00; B29C 65/16

(52) U.S. Cl. 600/101; 156/272.8

(57) ABSTRACT

A fitting section at which a cylindrical cover is fitted into a transparent cover, the cylindrical cover containing therein a CCD imager, and the transparent cover making visible light from a subject incident on the CCD imager, and being formed of a member transmitting laser light, is irradiated with laser light from the outside of an endoscope casing, thereby welding the cylindrical cover and the transparent cover together.

### CROSS-REFERENCE TO RELATED APPLICATIONS

This is a Continuation Application of PCT Application No. PCT/JP2006/325245, filed Dec. 19, 2006, which was published under PCT Article 21(2) in Japanese.

This application is based upon and claims the benefit of priority from prior Japanese Patent Application No. 2005-365209, filed Dec. 19, 2005, the entire contents of which are incorporated herein by reference.

### BACKGROUND OF THE INVENTION

1. Field of the Invention

The present invention relates to a small-sized capsule endoscope formed into a capsule-shape used for inspection or the like inside a living body, and a method of manufacturing the same.

2. Description of the Related Art

Techniques related to capsule endoscopes are disclosed in, for example, Jpn. Pat. Appln. KOKAI Publication No. 2001-91860 (paragraph No. [0012]), Jpn. Pat. Appln. KOKAI Publication No. 2004-65575 (paragraph Nos. [0028], [0071]), and Jpn. Pat. Appln. KOKAI Publication No. 2005-261504 (paragraph No. [0033]). In Jpn. Pat. Appln. KOKAI Publication No. 2001-91860, that a capsule endoscope includes an exterior case in which a circuit board and the like are encased in a watertight state, the exterior case is constituted of a substantially hemispheric transparent cover covering the front of an objective lens, and a cylindrical cover covering the rear of the objective lens and having a hemispheric shape at a rear end thereof, and the transparent cover and the cylindrical cover are bonded to each other in a watertight state, thereby forming a capsule endoscope is disclosed.

In Jpn. Pat. Appln. KOKAI Publication No. 2004-65575, that a capsule endoscope includes a capsule container sealed by using a front-end cover and a rear-end cover, an O-ring for watertight sealing is interposed between an outer circumferential surface of the main body and an inner circumferential surface of the rear cover, and the front-end cover constituted of a flexible material and the main body are fixed to each other by using an adhesive that can secure watertightness even when it is subjected to elastic deformation is disclosed.

In Jpn. Pat. Appln. KOKAI Publication No. 2005-261504, that an observation side cover and a capsule main body are fixed to each other by welding so as to be integral with each other by means of ultrasonic heat generation achieved by applying ultrasonic vibration to the cover and the main body is disclosed.

Pat. Document 1: Jpn. Pat. Appln. KOKAI Publication No. 2001-91860 (paragraph No. [0012])

Pat. Document 2: Jpn. Pat. Appln. KOKAI Publication No. 2004-65575 (paragraph No. [0028], [0071])

Pat. Document 3: Jpn. Pat. Appln. KOKAI Publication No. 2005-261504 (paragraph No. [0033])

### BRIEF SUMMARY OF THE INVENTION

In the techniques disclosed in Jpn. Pat. Appln. KOKAI Publication No. 2001-91860 and Jpn. Pat. Appln. KOKAI Publication No. 2004-65575, for example, when the transparent cover and the cylindrical cover are bonded to each other, it is difficult to achieve separation with constant application amount of adhesive. Further, in the technique disclosed in the above patent documents, when the transparent cover and the cylindrical cover are bonded to each other, an excessive amount of adhesive is applied to the outer surface of the capsule main body. An uneven part or a burr-like part is formed by the adhesive on the outer surface of the capsule main body in some cases. If such an uneven part or a burr-like part is formed, according to the above patent documents, complicated post-treatment process work must be performed in order to finish the outer surface of the capsule main body smooth.

The capsule endoscope is swallowed into a body of a person such as a patient, is used to inspect the inside of the living body, and is small-sized. It is undesirable for such a small-sized capsule endoscope that an uneven part or a burr-like part is formed at, for example, a bonded part at which the transparent cover and the cylindrical cover are bonded to each other. It is therefore necessary for the small-sized capsule endoscope that the bonding should be reliably performed with the outer surface of the capsule main body finished smooth.

The technique disclosed in Jpn. Pat. Appln. KOKAI Publication No. 2005-261504 can solve the problems of the above patent documents. However, in the technique of Jpn. Pat. Appln. KOKAI Publication No. 2005-261504, an ultrasonic horn is brought into contact with the observation side cover and the capsule main body, and an ultrasonic wave is oscillated. Therefore, there is the possibility of electronic components and the like contained in the capsule main body being adversely affected.

In the technique of Jpn. Pat. Appln. KOKAI Publication No. 2005-261504, the ultrasonic horn is brought into contact with the cover and the capsule main body, and hence a surface of a part of the resin which is brought into contact with the horn is heated. Thus, by the technique of the above patent document, the surface is roughened, and no smooth capsule outer surface can be obtained. Further, using the technique of the above patent document, it is difficult to achieve, for example, narrow and linear adhesion in the ultrasonic welding.

Besides the above problem, in order to bring the ultrasonic horn into contact with the observation side cover or the capsule main body, and effectively transmit the ultrasonic wave, there is the necessity to provide a notch or the like in the part of the cover or the capsule main body at which the ultrasonic horn is brought into contact with the cover or the capsule main body.

An object of the present invention is to provide a capsule endoscope in which welding and bonding can be securely performed by irradiation of laser light, and a method of manufacturing the capsule endoscope.

According to a first aspect of the present invention, there is provided a capsule endoscope comprising: a first exterior member formed of an optical resin member transmitting visible light and laser light; and a second exterior member which is arranged with respect to the first exterior member in such a manner that the second exterior member is brought into surface contact with the first exterior member, and is formed of a resin member containing a laser light absorbing agent, characterized in that laser light is applied to the second exterior member through the first exterior member, thereby melting the second exterior member, and the first exterior member and the second exterior member are bonded to each other by laser welding.

According to a second aspect of the present invention, a capsule endoscope comprising: a first exterior member formed of an optical resin member transmitting visible light and laser light; a second exterior member which is arranged with respect to the first exterior member in such a manner that the second exterior member is brought into surface contact with the first exterior member, and is formed of a resin member; and a laser light absorbing member provided at a part at which the first exterior member and the second exterior member are in surface contact with each other, characterized in that laser light is applied to the laser light absorbing member through the first exterior member, thereby melting the laser light absorbing member, and the first exterior member and the second exterior member are bonded to each other by laser welding.

According to a third aspect of the present invention, a method of manufacturing a capsule endoscope characterized by comprising: arranging a second exterior member formed of a resin member containing a laser light absorbing agent with respect to a first exterior member formed of an optical resin member transmitting visible light and laser light in such a manner that the second exterior member is brought into surface contact with the first exterior member; and applying laser light to the second exterior member through the first exterior member, thereby melting the second exterior member, and bonding the first exterior member and the second exterior member to each other by laser welding.

According to a fourth aspect of the present invention, a method of manufacturing a capsule endoscope characterized by comprising: arranging a second exterior member formed of a resin member with respect to a first exterior member formed of an optical resin member transmitting visible light and laser light in such a manner that the second exterior member is brought into surface contact with the first exterior member; providing a laser light absorbing member at a part at which the first exterior member and the second exterior member are in surface contact with each other; and applying laser light to the laser light absorbing member through the first exterior member, thereby melting the laser light absorbing member, and bonding the first exterior member and the second exterior member to each other by laser welding.

### BRIEF DESCRIPTION OF THE SEVERAL VIEWS OF THE DRAWING

FIG. 1 shows the configuration of a capsule endoscope according to a first embodiment of the present invention.

FIG. 2 shows the configuration of a capsule endoscope according to a second embodiment of the present invention.

FIG. 3 shows the configuration of a front-end section of a capsule endoscope according to a third embodiment of the present invention.

FIG. 4 shows the configuration of a front-end section of a capsule endoscope according to a fourth embodiment of the present invention.

FIG. 5 shows the configuration of a front-end section of an endoscope according to a fifth embodiment of the present invention.

FIG. 6 shows the configuration of a front end of an insertion section of an electronic endoscope according to a sixth embodiment of the present invention.

### DETAILED DESCRIPTION OF THE INVENTION

A first embodiment of the present invention will be described below with reference to the accompanying drawings.

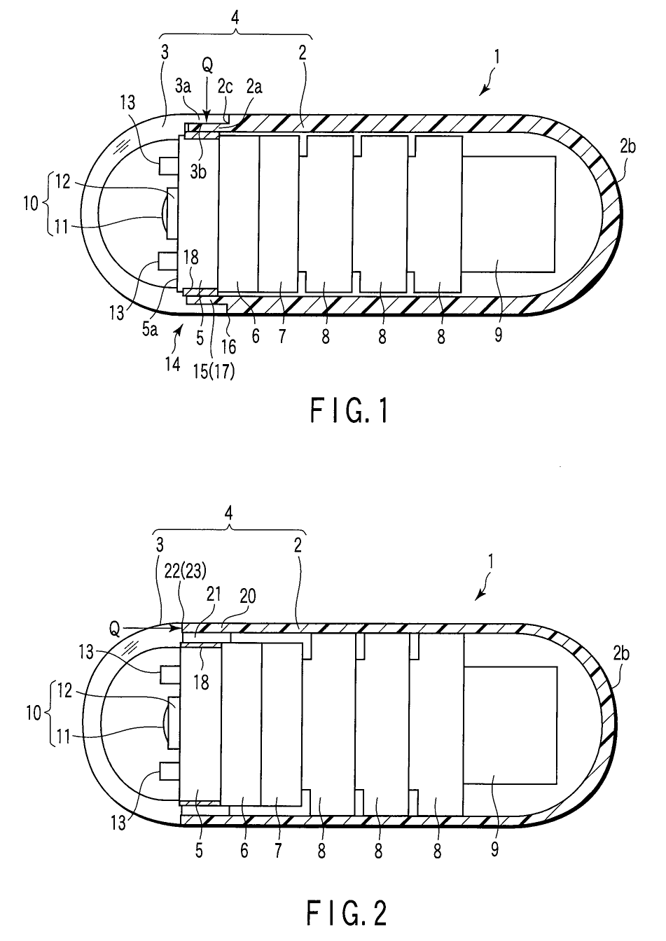

FIG. 1 shows a configuration view of a capsule endoscope. The capsule endoscope 1 is provided with a cylindrical cover 2 serving as a second exterior member, and a transparent cover 3 serving as a first exterior member. The cylindrical cover 2 and the transparent cover 3 are fitted to each other, thereby forming an integrated cylindrical capsule endoscope casing 4.

The cylindrical cover 2 is formed into a cylindrical shape. In the cylindrical cover 2, a circular first opening end section 2a is formed at one end part, and a hemispheric section 2b is formed at the other end part. The cylindrical cover 2 is formed of a resin member containing a laser light absorbing agent. That is, the cylindrical cover 2 is formed of a thermoplastic resin member which is a nontransparent resin member transmitting no laser light, and absorbs laser light (laser beam).

Types of the nontransparent resin member of the cylindrical cover 2 transmitting no laser light include, for example, polycarbonate (PC), ABC resin (ABC), polysulfone (PSU), polyphenylsulfone (PPSU), polyphenyleneoxide (PPO), polyphenylenesulfide (PPS), styrene resin, polyamide (PA) such as nylon 6 (PA6) and nylon 66 (PA66), polyethylene (PE), polypropylene (PP) and styrene-acrylonitrile copolymer, and the like, each of which is mixed with a predetermined coloring agent such as carbon black. Incidentally, the nontransparent resin member may be mechanically strengthened by glass fiber or the like as the need arises.

Further, a coloring member is added to the cylindrical cover 2. As a result, when the cylindrical cover 2 is melted by irradiation of laser light, the color of the cover 2 is changed by the coloring member. Examples of the coloring member include titanium white, TiO₂, a pigment (cyanine series), and the like.

The transparent cover 3 is formed into a hemispheric shape. A circular second opening end section 3a is formed at one end part of the transparent cover 3. The transparent cover 3 is formed of an optical resin member that transmits visible light and laser light. That is, the transparent cover 3 transmits white illuminating light, and transmits, for example, reflected light from a body cavity. The transparent cover 3 is formed of a light-transmitting resin member that transmits visible light, for example, 70% or more. Further, the transparent cover 3 is formed of a member which is a light-transmitting resin member having thermoplasticity. The light-transmitting resin member of the transparent cover 3 is not particularly limited as long as it is a member that transmits laser light at a predetermined transmissivity or more.

Types of the light-transmitting resin member include, for example, polycarbonate (PC), acrylic resin, cycloolefin polymer (COP), polyurethane (PU), styrene resin, and polyamide (PA) such as nylon 6 (PA6) and nylon 66 (PA66).

In the cylindrical cover 2, a CCD imager 5 serving as an image pickup device, a signal processing circuit 6, a communication processing circuit 7, a plurality of button-shaped batteries 8, and an antenna 9 are contained. Assuming that the transparent cover 3 side of the CCD imager 5 is the front side, the signal processing circuit 6, the communication processing circuit 7, the plural button-shaped batteries 8, and the antenna 9 are provided in the order mentioned from the rear side of the CCD imager 5.

An objective optical system 10 is provided on the transparent cover 3 side of the CCD imager 5. The objective optical system 10 forms light of an optical image transmitted through the transparent cover 3 and incident thereon into an image. The objective optical system 10 includes an objective lens 11, and an optical lens arranged in a lens frame 12. The CCD imager 5 is provided at an image formation position of the objective optical system 10. Further, around the objective optical system 10, a plurality of, for example, four white LEDs 13 are provided in the same plane as an illumination optical system. Incidentally, the objective optical system 10 and the white LEDs 13 are provided in a plane 5a on the transparent cover 3 side of, for example, the CCD imager 5.

The signal processing circuit 6 includes a circuit for emission-driving the white LEDs 13, a circuit for driving the CCD imager 5, and a circuit for forming an image pickup signal output from the CCD imager 5 into an image signal.

The communication processing circuit 7 transmits an image signal formed by the signal processing circuit 6 to an external apparatus as an electric wave.

The number of button-shaped batteries provided is, for example, three. These button-shaped batteries 8 supply power to the CCD imager 5, the signal processing circuit 6, the communication processing circuit 7, and the like.

The antenna 9 is provided in a hemispheric space formed by the hemispheric section 2b of the cylindrical cover 2. The antenna 9 is electrically connected to the communication processing circuit 7, and radiates a signal processed by the communication processing circuit 7 as an electric wave.

In the cylindrical cover 2 and the transparent cover 3, the first opening end section 2a of the cylindrical cover 2 is fitted into the second opening end section 3a of the transparent cover 3, thereby integrating the cylindrical cover 2 and the transparent cover 3 with each other. The first opening end section 2a is formed by providing a step 2c on the entire circumference on the outer surface side of the cylindrical cover 2. The second opening end section 3a is formed by providing a step 3b on the entire circumference on the inner surface side of the transparent cover 3.

However, the first opening end section 2a of the cylindrical cover 2 is inserted into the second opening end section 3a of the transparent cover 3, the second opening end section 3a being on the outside, and the first opening end section 2a being on the inside, thereby achieving the fit of the covers 2 and 3. In other words, the covers 2 and 3 are in a state where the transparent cover 3 covers the outer surface of the cylindrical cover 2.

This fitting section 14 includes a boundary surface 15 at which the second opening end section 3a and the first opening end section 2a are in surface contact with each other. The boundary surface is formed concentric with the side surface of the cylindrical capsule endoscope casing 4. Incidentally, a distal end part of the second opening end section 3a is hit against the step 2c of the cylindrical cover 2, and forms a hitting surface 16.

A laser welding section 17 is formed on the entire circumference of the cylindrical boundary surface 15. At the laser welding section 17, the first opening end section 2a and the second opening end section 3a are tightly and integrally bonded to each other in a watertight state by the welding caused by external irradiation of laser light Q.

That is, the laser light Q is transmitted through the second opening end section 3a of the transparent cover 3, and is applied to the first opening end section 2a of the cylindrical cover 2 formed of the thermoplastic resin member. The irradiation direction of the laser light Q is a direction substantially perpendicular to the transparent cover 3, i.e., a direction substantially perpendicular to the side surface of the capsule endoscope casing 4. In this case, the laser light Q is applied to the entire circumference of the side surface of the capsule endoscope casing 4 while the capsule endoscope casing 4 is rotated. Alternatively, the laser light Q is applied to the entire circumference of the side surface of the capsule endoscope casing 4 while the irradiation direction of the laser light Q is changed.

The laser light Q is transmitted through the transparent cover 3, and is applied to the cylindrical cover 2. As a result of this, the first opening end section 2a of the cylindrical cover 2 is heated and melted. Thus, the first opening end section 2a and the second opening end section 3a are thermally welded, i.e., the transparent cover 3 and the cylindrical cover 2 are welded together.

The laser light Q has such a wavelength that a predetermined transmissivity or more, for example, 26% or more can be obtained with respect to the transparent cover 3. As a result of this, when the laser light Q is transmitted through the transparent cover 3 formed of the light-transmitting resin member, the energy loss of the laser light Q is reduced.

As a result, the boundary surface 15 between the second opening end section 3a formed of the light-transmitting resin member and the first opening end section 2a formed of the thermoplastic resin member is irradiated with laser light Q with less energy loss. By the irradiation of the laser light Q, energy sufficient to heat and melt the boundary surface 15 is accumulated in the boundary surface 15. Thus, sufficient heating and melting take place at the boundary surface 15 and, thereafter the second opening end section 3a and the first opening end section 2a are welded together. That is, the cylindrical cover 2 and the transparent cover 3 are welded together by the laser welding section 17.

Further, a coloring member is added to the cylindrical cover 2. As a result, when the cylindrical cover 2 is melted by the irradiation of laser light, the color of the cover 2 is changed by the coloring member. However, when the cylindrical cover 2 and the transparent cover 3 are welded together by the laser welding section 17, the fact that the welding has been achieved can be confirmed by the change in color appearing on the cylindrical cover 2.

A protection member 18 is provided at the fitting section at which the first opening end section 2a is fitted into the second opening end section 3a. More specifically, the protection member 18 is provided on the entire circumference of the inner circumferential surface of the first opening end section 2a. The protection member 18 is provided on the entire circumference of the outer circumferential surface of the CCD imager 5. Incidentally, it is sufficient if the protection member 18 is provided on one of or both of the entire circumference of the inner circumferential surface of the first opening end section 2a, and the entire circumference of the outer circumferential surface of the CCD imager 5.

The protection member 18 absorbs the laser light Q applied to the boundary surface 15 between the second opening end section 3a and the first opening end section 2a. The protection member 18 absorbs scattered light produced when the laser light Q is applied. The protection member 18 is formed of, for example, a member such as ceramic excellent in heat insulating properties and light blocking effect. As described above, the protection member 18 absorbs the laser light Q and the scattered light thereof, and hence the CCD imager 5 and the like are not adversely affected by the laser light Q and the scattered light thereof.

As described above, according to the first embodiment, the capsule endoscope 1 includes the cylindrical cover 2 in which the CCD imager 5 is contained, and the transparent cover 3 which is formed of the member transmitting laser light, and through which visible light from the subject is made incident on the CCD imager 5, and the cylindrical cover 2 and the transparent cover 3 are welded together by irradiating the fitting section 14 at which the cylindrical cover 2 is fitted into the transparent cover 3 with the laser light Q from the outside of the capsule endoscope casing 4. As a result of this, even in a small-sized capsule endoscope, the cylindrical cover 2 and the transparent cover 3 can be reliably welded together, and no uneven part or burr-like part is formed on the outer surface of the capsule endoscope casing 4. The outer surface of the capsule endoscope casing 4 can therefore be maintained smooth. Furthermore, complicated post-treatment process work need not be performed.

The protection member 18 absorbs the laser light Q and the scattered light thereof. As a result of this, the laser light Q and the scattered light do not adversely affect the CCD imager 5 and the like.

Incidentally, the first embodiment may be modified as follows. In the first embodiment, as described above, the cylindrical cover 2 is formed of a resin member containing a laser light absorbing agent. That is, the cylindrical cover 2 is formed of a thermoplastic resin member, which is a nontransparent resin member that does not transmit laser light, and absorbs laser light. Further, when the laser light Q is transmitted through the transparent cover 3, and applied to the cylindrical cover 2, the cylindrical cover 2 is heated and melted, and the transparent cover 3 and the cylindrical cover 2 are welded together. However, the present invention is not limited to this. In the first embodiment, a laser light absorbing member may be provided at the part at which the cylindrical cover 2 and the transparent cover 3 are in surface contact with each other, and laser light Q may be applied through the transparent cover 3 to the laser light absorbing member, thereby melting the laser light absorbing member, and bonding the cylindrical cover 2 and the transparent cover 3 to each other by laser welding. The laser light absorbing member is formed of a thermoplastic resin member or an application agent that absorbs laser light. In this case, the cylindrical cover 2 need not contain a laser light absorbing agent.

Next, a second embodiment of the present invention will be described below with reference to the accompanying drawings. Incidentally, the same parts as those in FIG. 1 will be denoted by the same reference symbols and detailed description of them will be omitted.

FIG. 2 shows a configuration view of a capsule endoscope. A cylindrical cover 2 and a transparent cover 3 are made integral with each other by fitting a second opening end section 21 of the transparent cover 3 into a first opening end section 20 of the cylindrical cover 2. The first opening end section 20 is formed into a cylindrical shape. The second opening end section 21 is formed by providing a step 22 on the entire circumference on the outer surface side of the transparent cover 3.

However, the second opening end section 21 of the transparent cover 3 is inserted into the first opening end section 20 of the cylindrical cover 2, the first opening end section 20 being on the outside, and the second opening end section 21 being on the inside, thereby achieving the fit of the covers 2 and 3. In other words, the covers 2 and 3 are in a state where the cylindrical cover 2 covers the outer surface of the transparent cover 3. Further, a distal end part of the first opening end section 20 of the cylindrical cover 2 is in a state where it is hit against the step 22 of the transparent cover 3.

A laser welding section 23 is formed at a part at which the distal end part of the first opening end section 20 is hit against the step 22 of the transparent cover 3. That is, laser light Q is applied to the part at which the distal end part of the first opening end section 20 is hit against the step 22 of the transparent cover 3 in the same direction as the side surface direction of the capsule endoscope casing 4. In this case too, the laser light Q is applied to the circumferential hitting part while the capsule endoscope casing 4 is rotated. Alternatively, the laser light Q is applied to the circumferential hitting part while the irradiation position of the laser light Q is changed.

When the laser light Q is transmitted through the transparent cover 3, and is applied to the cylindrical cover 2, the distal end part of the first opening end section 20 of the cylindrical cover 2 is heated and melted. As a result of this, the transparent cover 3 and the cylindrical cover 2 are welded together.

As described above, according to the second embodiment, even when the laser welding section 23 is formed at the part at which the distal end part of the first opening end section 20 is hit against the step 22 of the transparent cover 3, the same effect as the first embodiment can be obtained. That is, even in a small-sized capsule endoscope, the cylindrical cover 2 and the transparent cover 3 can be reliably welded together, and an uneven part or a burr-like part is not formed on the outer surface of the capsule endoscope casing 4. The outer surface of the capsule endoscope casing 4 can therefore be maintained smooth. Furthermore, complicated post-treatment process work need not be performed.

Incidentally, in the second embodiment, a laser light absorbing member may be provided at the part at which the cylindrical cover 2 and the transparent cover 3 are in surface contact with each other, and laser light Q may be applied to the cylindrical cover 2, thereby melting the laser light absorbing member, and bonding the cylindrical cover 2 and the transparent cover 3 to each other by laser welding. The laser light absorbing member is formed of a thermoplastic resin member or an application agent that absorbs laser light. In this case, the cylindrical cover 2 need not contain a laser light absorbing agent.

Next, a third embodiment of the present invention will be described below with reference to the accompanying drawings.

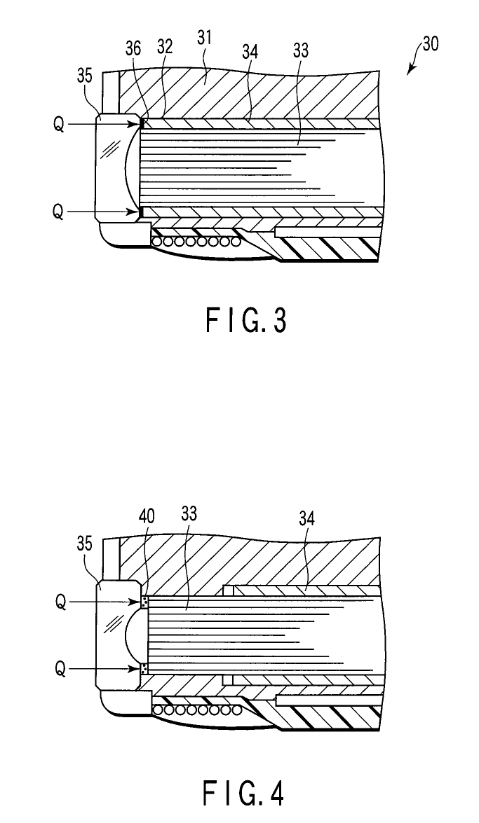

FIG. 3 shows a configuration view of an endoscope distal end section of a capsule endoscope. The endoscope distal end section 30 is a light guide part of the endoscope. The endoscope distal end section 30 is provided with a distal end constituent member 31 made of metal. The distal end constituent member 31 is provided with a hole 32 for attaching an illumination optical system. A light guide fiber bundle 33 is inserted in the hole 32 for attaching an illumination optical system, and fixed thereto. The light guide fiber bundle 33 is formed of a glass member. A pipe sleeve 34 is fixed to the light guide fiber bundle 33. The pipe sleeve 34 is formed of a thermoplastic resin member that absorbs laser light Q.

A cover glass 35 is inserted in a front end part of the hole 32 for attaching an illumination optical system. The cover glass 35 abuts on the pipe sleeve 34 at its distal end in a state where it is inserted in the hole 32 for attaching an illumination optical system. The cover glass 35 is formed of a light-transmitting resin member that transmits visible light and laser light Q.

A laser welding section 36 is formed at a part at which the cover glass 35 abuts on the pipe sleeve 34. The laser welding section 36 tightly and integrally bonds the cover glass 35 and the pipe sleeve 34 to each other, and fixes them to each other by welding caused by being irradiated with the laser light Q from outside. That is, the laser light Q is applied from the outside of the cover glass 35. The laser light Q is transmitted through the cover glass 35, and is applied to the abutment part between the cover glass 35 and the pipe sleeve 34. As a result of this, the cover glass 35 and the pipe sleeve 34 are heated and melted, thereby welding the cover glass 35 and the pipe sleeve together.

As described above, according to the third embodiment, the laser light Q is transmitted through the cover glass 35, and is applied to the abutment part between the cover glass 35 and the pipe sleeve 34. As a result of this, the cover glass 35 and the pipe sleeve 34 are heated and melted, thereby welding the cover glass 35 and the pipe sleeve 34 together. Thus, even in a small-sized endoscope distal end section, the cover glass 35 and the pipe sleeve 34 can be reliably welded together.

Next, a fourth embodiment of the present invention will be described below with reference to the accompanying drawings. Incidentally, the same parts as those in FIG. 3 will be denoted by the same reference symbols and detailed description of them will be omitted.

FIG. 4 shows a configuration view of an endoscope distal end section. A cover glass 35 is formed of a glass member. The cover glass 35 transmits visible light and laser light Q. A laser absorbing member 40 is provided between the cover glass 35 and a light guide fiber bundle 33. The laser absorbing member 40 is formed of a thermoplastic resin that absorbs laser light Q. Incidentally, a surface of the cover glass 35 in contact with the laser absorbing member 40 may be formed into, for example, an aventurine surface having a plurality of small spots.

Laser light Q is applied to the cover glass 35 from the outside thereof. The laser light Q is transmitted through the cover glass 35, and is applied to the laser absorbing member 40. As a result of this, the laser absorbing member 40 is heated and melted, thereby welding the cover glass 35 and the light guide fiber bundle 33 together. Incidentally, if the surface of the cover glass 35 in contact with the laser absorbing member 40 is formed into, for example, an aventurine surface having a plurality of small spots, the bond between the cover glass 35 and the light guide fiber bundle 33 becomes firm.

As described above, according to the fourth embodiment, the laser absorbing member 40 is provided between the cover glass 35 and the light guide fiber bundle 33, the laser light Q is applied to the laser absorbing member 40, thereby heating and melting the laser absorbing member 40, and welding the cover glass 35 and the light guide fiber bundle 33 together. As a result of this, even in a small-sized endoscope distal end section, the cover glass 35 and the pipe sleeve 34 can be reliably welded together.

Next, a fifth embodiment of the present invention will be described below with reference to the accompanying drawings. Incidentally, the same parts as those in FIG. 3 will be denoted by the same reference symbols and detailed description of them will be omitted.

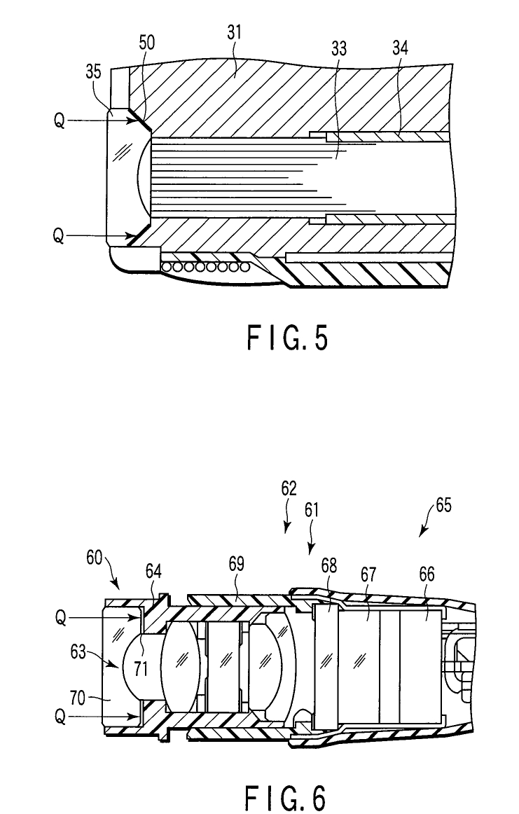

FIG. 5 shows a configuration view of an endoscope distal end section. A distal end constituent member 31 is formed of a thermoplastic resin member that absorbs laser light Q. The distal end constituent member 31 is provided with a tapered opening section 50. The tapered opening section 50 is formed into a tapered shape that spreads out from the side on which the light guide fiber bundle 33 is provided toward the opened side. A caliber of the tapered opening section 50 is formed larger than a diameter of the light guide fiber bundle 33.

A cover glass 35 is provided in the tapered opening section 50. The cover glass 35 can have a diameter larger than that of the cover glass 35 used in FIG. 3 or 4. A surface of the cover glass 35 in contact with the tapered opening section 50 is formed into an inclined (tapered) surface that can be fitted in the tapered shape of the tapered opening section 50. The cover glass 35 is formed of a light-transmitting resin member that transmits visible light and laser light.

When laser light Q is applied from the outside of the cover glass 35, the laser light Q is transmitted through the cover glass 35, and is applied to an abutment part between the cover glass 35 and the tapered opening section 50 of the distal end constituent member 31. As a result of this, the cover glass 35 and the tapered opening section 50 are heated and melted, thereby welding the cover glass 35 and the tapered opening section 50 together.

As described above, according to the fifth embodiment, the distal end constituent member 31 is provided with the tapered opening section 50, and the cover glass 35 is welded to the tapered opening section 50. As a result of this, even in a small-sized endoscope distal end section, the cover glass 35 can be reliably welded to the tapered opening section 50.

Incidentally, the third to fifth embodiments described above are not limited to the light guide part of the endoscope, and can also be applied to an image guide.

Next, a sixth embodiment of the present invention will be described below with reference to the accompanying drawings.

FIG. 6 shows a configuration view of an insertion section distal end of an electronic endoscope. The electronic endoscope insertion section distal end is constituted of a distal end constituent section formed of a hard member and a distal end insulating cover for covering the distal end constituent section. Of these members, the distal end constituent section is formed of a metallic member or the like, and is formed into a substantially pillar-like shape.

The distal end constituent section includes an imaging unit formed by integrating a first unit 60 and a second unit 61 into one unit. In the first unit 60, an objective lens 63 which is arranged at the front end, and is constituted of a plurality of optical systems is arranged in a lens frame 64 which is a first frame body, whereby an objective unit is formed.

The second unit 61 is provided behind the first unit 60. The second unit 61 includes an imaging section 65 for imaging an image made incident thereon through the objective unit. The imaging section 65 is formed by arranging a CCD chip 66, a first cover glass 67, a second cover glass 68, and the like. In the second unit 61, a channel hole or the like for arranging a forceps channel member is formed. The forceps channel member constitutes a unit insertion hole, and treatment tool insertion channel. In the unit insertion hole, the imaging unit 62 is arranged. In the treatment tool insertion channel, a treatment tool such as biopsy forceps or the like is inserted.

The second unit 61 includes a CCD holder 69 which is a second frame body. The CCD holder 69 is fitted on the rear side of the lens frame 64 of the first unit 60. The CCD holder 69 is provided on a front end surface of the imaging section 65. By fitting the CCD holder 69 on the lens frame 64, the first unit 60 and the second unit 61 are integrated into one unit, i.e., the imaging unit 62.

The objective lens 63 includes a lens 70. The lens 70 is formed of a resin member. The lens frame 64 is formed of a resin member. Accordingly, both the lens 70 and the lens frame 64 are constituted of a combination of resin members.

A resin member 71, which is a laser light absorbing member, is provided between the lens 70 and the lens frame 64.

When laser light Q is applied from the outside of the objective lens 63, the laser light Q is transmitted through the lens 70 of the objective lens 63, and is applied to the resin member 71 provided between the lens 70 and the lens frame 64. As a result of this, the lens 70 and the lens frame 64 are welded together by the melted resin member 71.

Incidentally, each of a surface of the lens 70 and a surface of the lens frame 64 which are in contact with the resin member 71 may be formed into, for example, an aventurine surface having a plurality of small spots. This makes the bond between the lens 70 and the lens frame 64 firm.

As described above, according to the sixth embodiment, the resin member 71 is provided between the lens 70 and the lens frame 64, the resin member 71 is irradiated with the laser light Q, thereby welding the lens 70 and the lens frame 64 together by the thus melted resin member 71. As a result of this, even in a small-sized insertion section distal end of an electronic endoscope, the lens 70 and the lens frame 64 can be reliably welded together.

The above description has been given of an example in which the resin member 71 is provided and, if the lens frame 64 is formed of a laser light absorbing member, the resin member 71 may be omitted.

What is claimed is:

1. A capsule endoscope comprising:
a first exterior member formed of an optical resin member transmitting visible light and laser light; and
a second exterior member which is arranged with respect to the first exterior member in such a manner that the second exterior member is brought into surface contact with the first exterior member, and is formed of a resin member containing a laser light absorbing agent, wherein
laser light is applied to the second exterior member through the first exterior member, thereby melting the second exterior member, and the first exterior member and the second exterior member are bonded to each other by laser welding.

2. A capsule endoscope comprising:
a first exterior member formed of an optical resin member transmitting visible light and laser light;
a second exterior member which is arranged with respect to the first exterior member in such a manner that the second exterior member is brought into surface contact with the first exterior member, and is formed of a resin member; and
a laser light absorbing member provided at a part at which the first exterior member and the second exterior member are in surface contact with each other, wherein
laser light is applied to the laser light absorbing member through the first exterior member, thereby melting the laser light absorbing member, and the first exterior member and the second exterior member are bonded to each other by laser welding.

3. The capsule endoscope according to claim 1 or 2, wherein
a capsule endoscope casing formed by bonding the first exterior member and the second exterior member to each other is formed into a cylindrical shape, and
a bonded part at which the first exterior member and the second exterior member are bonded to each other is provided on the entire circumference of a side surface of the capsule endoscope.

4. The capsule endoscope according to claim 1 or 2, wherein
in a bonded part at which the first exterior member and the second exterior member are bonded to each other, the second exterior member is fitted into the first exterior member, the first exterior member being on the outside, and the second exterior member being on the inside.

5. The capsule endoscope according to claim 1 or 2, further comprising:
a first opening end section formed in the first exterior member; and
a second opening end section formed in the second exterior member, wherein
in a bonded part at which the first exterior member and the second exterior member are bonded to each other, the second opening end section is inserted/fitted into the first opening end section, and at a boundary surface between the first opening end section and the second opening end section, the first opening end section and the second opening end section are integrally and tightly fixed to each other by welding achieved by laser light irradiation.

6. The capsule endoscope according to claim 5, wherein
the bonded part at which the first exterior member and the second exterior member are bonded to each other includes a fitting surface at which the first opening end section and the second opening end section are in surface contact with each other, and a hitting surface at which the first opening end section and the second opening end section hit against each other, and is formed by welding achieved by irradiating at least one of the fitting surface and the hitting surface with the laser light.

7. The capsule endoscope according to claim 1 or 2, wherein the second exterior member is formed of a thermoplastic resin member.

8. The capsule endoscope according to claim 1 or 2, wherein the second exterior member contains a coloring member.

9. The capsule endoscope according to claim 2, wherein the laser light absorbing member is provided on an inner wall surface of the first exterior member.

10. The capsule endoscope according to claim 2, wherein the laser light absorbing member is provided on an outer wall surface of the second exterior member.

11. The capsule endoscope according to claim 1 or 2, wherein the laser light is provided with a transmissivity equal to 26% or more with respect to the first exterior member.

12. The capsule endoscope according to claim 1 or 2, further comprising a protection member which is provided at a fitting section at which the end section of the second exterior member is fitted into the end section of the first exterior member, and absorbs at least the laser light.

13. The capsule endoscope according to claim 3, further comprising a protection member which is provided at a fitting section at which the end section of the second exterior member is fitted into the end section of the first exterior member, and absorbs at least the laser light.

14. A method of manufacturing a capsule endoscope comprising:
arranging a second exterior member formed of a resin member containing a laser light absorbing agent with respect to a first exterior member formed of an optical resin member transmitting visible light and laser light in such a manner that the second exterior member is brought into surface contact with the first exterior member; and
applying laser light to the second exterior member through the first exterior member, thereby melting the second exterior member, and bonding the first exterior member and the second exterior member to each other by laser welding.

15. A method of manufacturing a capsule endoscope comprising:
arranging a second exterior member formed of a resin member with respect to a first exterior member formed of an optical resin member transmitting visible light and laser light in such a manner that the second exterior member is brought into surface contact with the first exterior member;
providing a laser light absorbing member at a part at which the first exterior member and the second exterior member are in surface contact with each other; and
applying laser light to the laser light absorbing member through the first exterior member, thereby melting the laser light absorbing member, and bonding the first exterior member and the second exterior member to each other by laser welding.
